(19) United States
(12) Patent Application Publication
(10) Pub. No.: US 2006/0088442 A1
(43) Pub. Date: Apr. 27, 2006

(54) Chemical thermal desorption system

(75) Inventors: Joel Del Eckels, Livermore, CA (US); Carolyn Koester, Pleasanon, CA (US); Armando Alcaraz, Livermore, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 10/974,211

(22) Filed: Oct. 26, 2004

(51) Int. Cl. C12Q 1/68

(52) U.S. Cl. 422/50

(57) ABSTRACT

A field portable chemical thermal desorption system. The system comprises a desorption tube, an injection needle operatively connected to the desorption tube, a needle valve operatively connected to the injection needle, a heater operatively connected to the desorption tube, heater controller operatively connected to the heater, a gas supply operatively connected to the desorption tube, and a pressure regulator operatively connected to the gas supply.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to chemical thermal desorption and more particularly to a chemical thermal desorption system.

2. State of Technology

U.S. Pat. No. 5,092,218 issued Mar. 3, 1992 provides the following state of technology information, “Detection of explosives carried by persons or concealed in buildings, baggage, airplanes, cars or other locations can be vital to prevention of injuries and damage to property. However, detection by direct searching is quite costly and time-consuming, can at times be dangerous, and can also be susceptible to error. Thus, it is desirable to detect explosives somewhat indirectly, as by their presence in very small amounts of vapors in air or other gases which have been in contact with explosives in solid or liquid form.” (Col. 1, lines 17-26, U.S. Pat. No. 5,092,218)

U.S. Pat. No. 4,976,924 issued Dec. 11, 1990 provides the following state of technology information, “The trapping and collection of airborne contaminants in the form of gases, vapours, or the like is of importance in a number of fields. For example, contaminants may be collected to determine the downwind spread of herbicides or pesticides from a crop spraying operation to determine industrial compliance with air quality standards, to monitor the workplace air quality, or to control the use of toxic materials and to monitor and identify toxic substances released during military encounters. The current techniques for collecting air samples are:

a. by using liquid filled impingers or bubblers. Air is drawn (bubbled) through the liquid which partially, or completely, dissolves the contaminants and thus removes them from the air stream. The solution is subsequently analyzed by injection into, e.g., a gas chromatograph;

b. by using tubes packed with a solid porous adsorbant. The solid material retains contaminants by, e.g., physical inclusion in pores or electrostatic attraction at active polar sites on the surface of the solid when air is drawn through the tubes. The solid material is subsequently solvent extracted and the extract is analyzed, or the material is heated to thermally desorb the trapped contaminants into an appropriate apparatus for analysis;

c. by collecting sample volumes of the air in suitable containers such as large bags or gas syringes. The air is withdrawn from the collection container using a small gas-tight syringe and injected into an analysis apparatus;

d. by drawing air continuously through analyzers which monitor contaminant concentration in real time or near real time.” (Col. 1, lines 15-59, U.S. Pat. No. 4,976,924)

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

Currently available commercial thermal desorption units are large and not suited for field portable use. The present invention provides a chemical thermal desorption system that is field portable. The field portable chemical thermal desorption system comprises a desorption tube, an injection needle operatively connected to the desorption tube, a needle valve operatively connected to the injection needle, a heater operatively connected to the desorption tube, heater controller operatively connected to the heater, a gas supply operatively connected to the desorption tube, and a pressure regulator operatively connected to the gas supply. The field portable chemical thermal desorption system provides a thermal desorption system for the collection and desorption of chemicals of interest for analysis by gas chromatograph (GC), gas chromatograph-mass spectrometer (GCMS), and other instruments. The field portable chemical thermal desorption system utilizes a carrier gas and a heater system that allows direct injection into analytical instruments. The desorption tube contains an absorbent medium for capturing chemicals of interest and retaining these chemicals until heated. The gas supply provides gas which flows through the desorption tube and through the injection needle to transport the desorbed material into the gas chromatograph, gas chromatograph-mass spectrometer, or other instrument into which the injection needle has been placed.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates field portable chemical thermal desorption system constructed in accordance with the present invention.

FIG. 2 shows a field portable embodiment of the present invention with a field portable gas chromatography-mass spectrometry unit.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

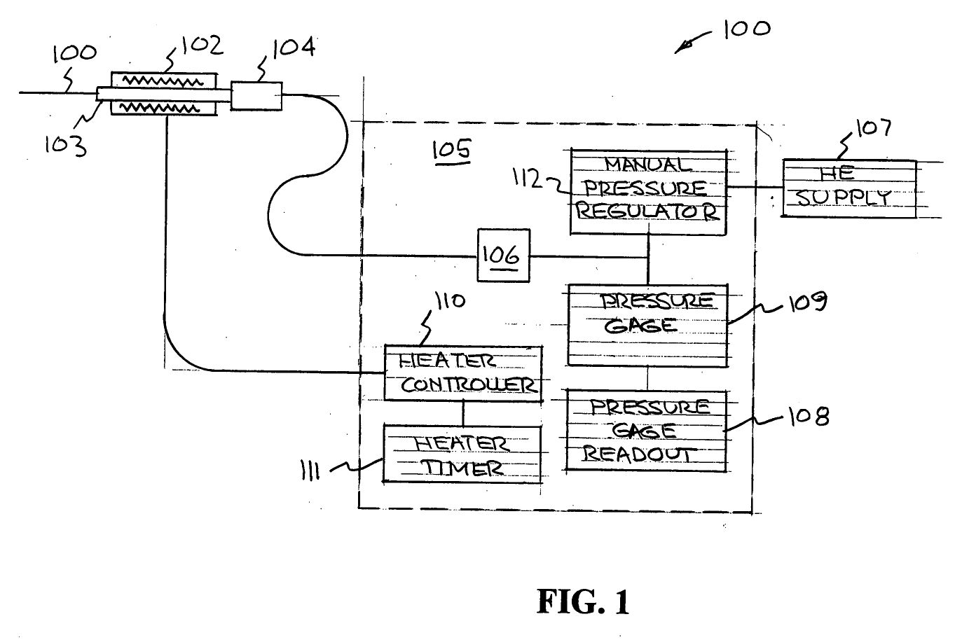

Referring now to FIG. 1, a field portable chemical thermal desorption system constructed in accordance with the present invention is illustrated. The field portable chemical thermal desorption system is designated generally by the reference numeral 100.

Currently available commercial thermal desorption units are large and not suited for field portable use. The chemical thermal desorption system 100 is field portable as a result of miniaturization and portability features that have been incorporated into the system. The field portable chemical thermal desorption system 100 utilizes carrier gas control and heater control systems that will allow direct injection into analytical instruments. The system 100 includes a number of distinct components. These components include injection needle 101, clam shell heater 102, desorption tube 103, slip on connector 104, control box 105, needle valve 106, helium gas supply 107, pressure gage readout 108, pressure gage 109, heater controller 110, heater timer 111, and manual pressure regulator 112.

The field portable chemical thermal desorption system 100 provides a thermal desorption system for the collection and desorption of chemicals of interest for analysis by gas chromatograph (GC), gas chromatograph-mass spectrometer (GCMS), and other instruments. The field portable chemical thermal desorption system 100 provides a relatively quick field analysis of various explosives and their chemical components.

As illustrated in FIG. 1, the syringe like needle 101 is attached by screw threads to the desorption tube 103. The clam shell heater 102 is placed immediately around the desorption tube 103 and is controlled by the temperature controller 110, and the timing control 111. This allows for rapid temperature rise of the medium contained in the desorption tube for a specific length of time. Helium gas is supplied to the desorption tube 103 via the slip-on connection 104 which is connected to the helium carrier gas control box 105. Gas flow and pressure are read out and controlled by manually setable components 108, 109, 106, as well as the manual pressure regulator 112.contained within the control box 105. The helium gas supply 107 provides carrier gas to the system.

The desorption tube 103 contains an absorbent medium suitable for capturing chemicals of interest and retaining these chemicals until a heating sequence on this desorption tube releases the chemicals previously captured. During this desorption phase, the carrier gas, usually helium, flows through the desorption tube 103 and through the injection needle 101 to transport the desorbed material into a gas chromatograph into which the injection needle has been placed.

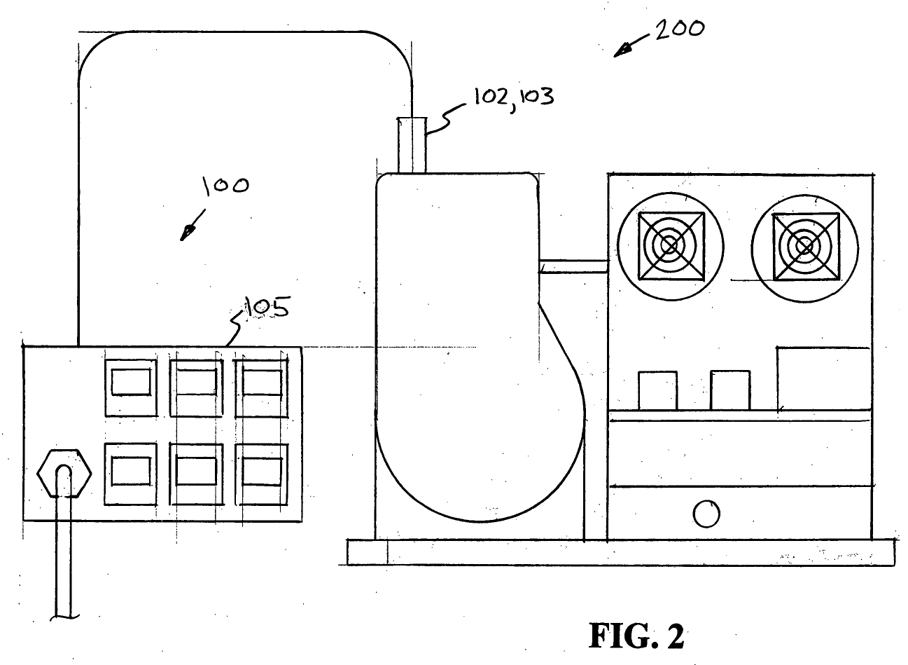

Referring now to FIG. 2, a field portable embodiment of the present invention is illustrated with a field portable gas chromatography-mass spectrometry (GC-MS) unit. The overall illustration is designated generally by the reference numeral 200. The illustration 200 shows the field portable chemical thermal desorption system 100 operatively connected to a field portable GC-MS unit 200.

The field portable GC-MS unit 200 can for example be a Constellation Technology Corporation Field Portable GC-MS unit. The Constellation Technology Corporation Field Portable GC-MS unit is ideal for identifying low-level amounts of CW agents and other volatile, highly toxic substances is gas chromatography-mass spectrometry (GC-MS). This analytical tool has the sensitivity required for performing such low-level analyses, and the ability to provide irrefutable confirmation of the identity of a volatile or semi-volatile substance, including CW agents. The Constellation field-portable CT-1128 GC-MS unit weighs 75 pounds and provides reliable data of laboratory quality in approximately 17 minutes. The unit is fully capable of detecting a wide variety of volatile and semi-volatile compounds including, but not limited to, CW agents and their surrogates, controlled substances (i.e. illicit drugs), explosives, and industry-related solvents and chemicals. The field portable GC-MS unit 201 can be used with other analysis systems.

As illustrated in FIG. 2, the field portable chemical thermal desorption system 100 operatively connected to the field portable GC-MS unit 200. The desorption tube 103 contains an absorbent medium suitable for capturing chemicals of interest and retaining these chemicals until a heating sequence on this desorption tube releases the chemicals previously captured. During this desorption phase, the carrier gas, usually helium, flows through the desorption tube 103 and through the injection needle to transport the desorbed material into the gas chromatograph 200 into which the injection needle has been placed.

Prior to introduction into the thermal desorption unit 100, explosive particles/vapors have been transferred into a sorbent-filled tube using heat and a flow of inert gas. This sorbent-filled tube containing analytes is then placed in the thermal desorption unit 100. A flow of heat and inert gas is used to transfer the analytes from the sorbent-filled tube 100 and into the Constellation gas chromatographic mass spectrometer (GC/MS) 200. Once transferred into the GC/MS 200, the analytes are separated by gas chromatography, ionized, and detected by mass spectrometry.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A field portable chemical thermal desorption apparatus, comprising:
a desorption tube,
an injection needle operatively connected to said desorption tube,
a needle valve operatively connected to said injection needle,
a heater operatively connected to said desorption tube,
heater controller operatively connected to said heater,
a gas supply operatively connected to said desorption tube, and
a pressure regulator operatively connected to said gas supply.

2. The field portable chemical thermal desorption apparatus of claim 1 wherein said gas supply is a helium gas supply.

3. The field portable chemical thermal desorption apparatus of claim 1 wherein said heater is a clam shell heater.

4. The field portable chemical thermal desorption apparatus of claim 1 wherein said heater is a clam shell heater and wherein said clam shell heater is placed immediately around said desorption tube.

5. The field portable chemical thermal desorption apparatus of claim 1 wherein said desorption tube contains an absorbent medium for capturing chemicals of interest and retaining these chemicals until heated.

6. The field portable chemical thermal desorption apparatus of claim 1 wherein said gas supply flows through said desorption tube and through said injection needle to transport the desorbed material.

7. The field portable chemical thermal desorption apparatus of claim 1 wherein said gas supply flows through said desorption tube and through said injection needle to transport the desorbed material into a gas chromatograph, a gas chromatograph-mass spectrometer, or other instrument into which said injection needle has been placed.

8. The field portable chemical thermal desorption apparatus of claim 1 wherein said desorption tube contains an absorbent medium for capturing chemicals of interest and retaining these chemicals until heated and wherein said gas supply flows through said desorption tube and through said injection needle to transport the desorbed material into a gas chromatograph, a gas chromatograph-mass spectrometer, or other instrument into which said injection needle has been placed.

9. A field portable chemical thermal desorption apparatus, comprising:
desorption tube means for capturing chemicals of interest,
injection needle means for providing flow, said injection needle means operatively connected to said desorption tube means,
heater means for heating said desorption tube means, said heater means operatively connected to said desorption tube means, and
gas supply means for providing flow of gas through said desorption tube means and said injection needle means, said gas supply means
operatively connected to said desorption tube means and said injection needle means.

10. The field portable chemical thermal desorption apparatus of claim 9 wherein said gas supply means is a helium gas supply.

11. The field portable chemical thermal desorption apparatus of claim 9 wherein said heater means is a clam shell heater.

12. The field portable chemical thermal desorption apparatus of claim 9 wherein said heater means is a clam shell heater and wherein said clam shell heater is placed immediately around said desorption tube means.

13. The field portable chemical thermal desorption apparatus of claim 9 wherein said desorption tube means includes an absorbent material for capturing chemicals of interest and retaining these chemicals of interest until heated.

14. The field portable chemical thermal desorption apparatus of claim 9 wherein said gas supply means provides helium gas which flows through said desorption tube means and through said injection needle means to transport desorbed material into a gas chromatograph, a gas chromatograph-mass spectrometer, or other instrument.

15. The field portable chemical thermal desorption apparatus of claim 9 wherein said gas supply means provides helium gas which flows through said desorption tube means and said desorption tube means contains an absorbent medium for capturing chemicals of interest and retaining these chemicals until heated and wherein said helium gas flows through said desorption tube means and through said injection needle means to transport said chemicals of interest into a gas chromatograph, a gas chromatograph-mass spectrometer, or other instrument.
